(19) United States
(12) Patent Application Publication
(10) Pub. No.: US 2024/0065604 A1
(43) Pub. Date: Feb. 29, 2024

(54) DIRECTIONAL AND SCALABLE ELECTRODE ARRAY

(71) Applicant: BOARD OF REGENTS OF THE UNIVERSITY OF TEXAS SYSTEM, Austin, TX (US)

(72) Inventors: John P. SEYMOUR, Houston, TX (US); Nitin TANDON, Houston, TX (US)

(21) Appl. No.: 18/250,532

(22) PCT Filed: Oct. 26, 2021
(86) PCT No.: PCT/US21/56661

§ 371 Date: Apr. 25, 2023

Related U.S. Application Data
(60) Provisional application No. 63/105,403, filed on Oct. 26, 2020.

(51) Int. Cl. A61N 1/05

(52) U.S. Cl. CPC A61N 1/0534

(57) ABSTRACT

A directional and scalable (DISC) electrode array includes an insulating body, a first plurality of microelectrodes, and a second plurality of microelectrodes. The insulating body includes an electrically insulating material, and has a length and a diameter. The diameter is at least 400 microns, and the length is greater than the diameter. The first plurality of microelectrodes is disposed along the length of the insulating body. The second plurality of microelectrodes is disposed along the length of the insulating body opposite the first plurality of microelectrodes. Further columns of microelectrodes improve the directional sensitivity of DISC.

### CROSS-REFERENCE TO RELATED APPLICATIONS

The present application claims priority to U.S. Provisional Patent Application No. 63/105,403, filed Oct. 26, 2020, entitled “Directional and Scalable Electrode Array,” which is hereby incorporated herein by reference in its entirety.

### BACKGROUND

In the human body, electrical signals are transmitted via the nervous system to and from the brain. For example, various body subsystems, such as sensory organs, generate electrical signals that are transmitted to the brain via the nervous system. Similarly, the brain generates electrical signals for controlling muscles and other body systems. A variety of sensing devices have been developed to interface with neural tissue and detect the electrical signals propagated within the neural tissue.

### SUMMARY

A directional and scalable (DISC) electrode array that provides a number of advantages over conventional neural sensors is described herein. In one example, a DISC electrode array includes an insulating body, a first plurality of microelectrodes, and a second plurality of microelectrodes. The insulating body includes an electrically insulating material, and has a length and a diameter. The diameter is at least 400 microns, and the length is greater than the diameter. The first plurality of microelectrodes is disposed along the length of the insulating body. The second plurality of microelectrodes is disposed along the length of the insulating body opposite the first plurality of microelectrodes.

An embodiment of the DISC electrode array may include a third plurality of microelectrodes disposed along the length of the insulating body orthogonal to the first plurality of microelectrodes and the second plurality of microelectrodes. An embodiment of the DISC electrode array may also include a fourth plurality of microelectrodes disposed along the length of the insulating body opposite the third plurality of microelectrodes. An embodiment of the DISC electrode array may also include a fifth plurality of microelectrodes and a sixth plurality of microelectrodes disposed along the length of the insulating body and opposite to each other. An embodiment of the DISC electrode array may also include a seventh plurality of microelectrodes and an eighth plurality of microelectrodes disposed along the length of the insulating body opposite to each other, and placed orthogonal to the fifth plurality of microelectrodes and the sixth plurality of microelectrodes. In an embodiment of the DISC electrode array, the diameter of the insulating body is in a range of 400 microns to 2000 microns. In one embodiment of the DISC electrode array, the diameter of the insulating body is approximately 800 microns.

In an embodiment of the DISC electrode array, the length of the insulating body is in a range of 3 millimeters to 150 millimeters. In an embodiment of the DISC electrode array, the microelectrodes have a diameter of at least 10 microns. Some embodiments of the DISC electrodes have a diameter of no more than 400 microns. In an embodiment of the DISC electrode array, two adjacent microelectrodes of the first plurality of microelectrodes are spaced apart in a range of 200-600 microns and are electrically independent from adjacent electrodes. In an embodiment of the DISC electrode array, the insulating body is cylindrical. In an embodiment of the DISC electrode array, the insulating body is polygonal in cross section.

In another example, a method for using a DISC electrode array includes acquiring first neuroelectric signals via a first plurality of microelectrodes disposed along a length of an insulating body formed of an electrically insulating material. The method also includes acquiring second neuroelectric signals via a second plurality of microelectrodes disposed opposite the first plurality of microelectrodes along the length of the insulating body. The method further includes providing the first neuroelectric signals and the second neuroelectric signals to a processing system for interpretation of neural activity.

An embodiment of the method may also include 1) acquiring third neuroelectric signals via a third plurality of microelectrodes disposed along the length of the insulating body orthogonal to the first plurality of microelectrodes and the second plurality of microelectrodes; 2) acquiring fourth neuroelectric signals via a fourth plurality of microelectrodes disposed opposite the third plurality of microelectrodes along the length of the insulating body; and 3) providing the third neuroelectric signals and the fourth neuroelectric signals to the processing system for interpretation of neural activity. An embodiment of the method may also include: 1) selecting the neuroelectric signals acquired by the first plurality of microelectrodes, the second plurality of microelectrodes, the third plurality of microelectrodes, and the fourth plurality of microelectrodes disposed along a selected length of the DISC electrode array; and 2) combining the selected neuroelectric signals to simulate output of a ring electrode if so desired, such as averaging local electrodes to lower independent noise sources. Another combination of local electrodes will primarily be along the length and only partially along the radial axis.

An embodiment of the method may also include: 1) selecting the neuroelectric signals acquired by a single microelectrode of the first plurality of microelectrodes; and 2) sensing a local field potential source (e.g., 0.1 to 300 Hz) based on the neuroelectric signals acquired by the single microelectrode.

In a further example, a method for fabricating a directional and scalable (DISC) electrode array includes securing a first linear array of microelectrodes to an insulating body, and securing a second linear array of microelectrodes to the insulating body opposite the first linear array of microelectrodes. An embodiment of the method may also include applying an adhesive to one or more of the first linear array of microelectrodes or the insulating body. An embodiment of the method may also include wrapping a thin film array of microelectrodes around the insulating body to form the first linear array of microelectrodes and the second linear array of microelectrodes. An embodiment of the method may also include: 1) positioning the first linear array of microelectrodes in a mold; 2) positioning the second linear array of microelectrodes in the mold opposite the first linear array of microelectrodes; and 3) injecting an insulating material into the mold. Another embodiment of the method may also include: 1) positioning a single multi-column array of electrodes in a mold; and 2) injecting an insulating material into the mold resulting in multiple columns and rows of microelectrodes. An embodiment of the method may also include additive manufacturing of the conductors, substrate, electrodes, and connection pads. An embodiment of the method may also include: 1) printing an insulating substrate onto a sacrificial cylinder; 2) printing a plurality of conductors and electrodes, and 3) printing an outer insulating layer to electrically isolate the plurality of conductors.

### BRIEF DESCRIPTION OF THE DRAWINGS

For a detailed description of various examples, reference will now be made to the accompanying drawings in which:

FIG. 1 shows an example directional and scalable (DISC) electrode array.

FIGS. 2A-2C illustrate directionality and amplitude of as a function of substrate diameter. FIG. 2C summarizes directionality for an insulator (darker shade) and for a hypothetical substrate with the same conductivity as surrounding tissue (lighter shade). Inset A refers back to FIG. 2A and inset B refers back to FIG. 2B.

FIG. 3 illustrates normalized voltage amplitude as a function of electrode diameter. Sensitivity to a generic distant dipole decreases as shown with increasing electrode diameter until finally a ring is formed and the amplitude reduction is a step function.

FIGS. 4A-4E show an electro-quasistatic 3D dipole model demonstrating directional sensitivity of a DISC electrode array in a multi-source configuration. FIGS. 4A and 4B show the geometric orientation of 8 sources (only half of a dipole source is shown) and the resulting superimposed potential when all sources are activated at maximum current density. FIG. 4C illustrates the ability to detect source 1 for 1 trial up to 50 repeated trials. CAR is the acronym for common average referencing. SNR is higher initially and increases at a faster rate with repeated trials. FIG. 4E shows the SNR results at 1 (lighter shade) and 50 trials (darker shade) for all 8 sources in this multi-source model.

FIGS. 5A-5C illustrate directional sensitivity of a DISC electrode array. In one example experiment, FIG. 5A shows, the location of the DISC electrode array relative to neural barrel cortex sources is identified. In FIG. 5B, the predicted magnitude, direction, and profile of each distinct neural source is shown as an example of source separation.

FIG. 5C is an example of using the laminar information available in some uses of DISC to provide multi-directional current source density analysis.

FIG. 6 shows a schematic cross-section of polyimide linear electrode (PLE) array suitable for use in a DISC electrode array.

FIG. 7 shows a micrograph of an example strip of single-column microelectrodes suitable for use in a DISC electrode array.

FIG. 8 illustrates examples of scalability achievable using various combinations of microelectrode referencing schemes of the DISC electrode array of FIG. 1 to produce improved sensitivity in various volumes of surrounding tissue.

FIG. 9 shows an example DISC electrode array implanted for use as a translatable human brain-computer interface.

FIGS. 10A-10B illustrate example methods for fabricating a DISC electrode array.

### DETAILED DESCRIPTION

Certain terms have been used throughout this description and claims to refer to particular system components. As one skilled in the art will appreciate, different parties may refer to a component by different names. This document does not intend to distinguish between components that differ in name but not function. In this disclosure and claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections. The recitation “based on” is intended to mean “based at least in part on.” Therefore, if X is based on Y, X may be a function of Y and any number of other factors.

Various sensor technologies for detecting electrical signals in neural tissue are available. Microwires, stereo-electroencephalogram (sEEG) depth electrodes, and electrocorticography (ECoG) electrodes (also known as grid arrays) are examples of conventional neural sensing technologies. These sensor technologies are subject to a number of shortcomings. For example, microwires and ring electrodes in sEEG, ECoG, and local field potential (LFP), record from millions of neurons over long distances, especially in the cortex of mammals including humans. The signals from overlapping neural input for any brain network—say speech production—are difficult to deconvolve, rendering sub-optimal decoding of phonological or articulatory codes. Implanting of grid electrodes can result in hemorrhaging, infections, and/or migraine headaches. Relative to craniotomies of several inches or larger, sEEG methods reduce these 3 important adverse events.

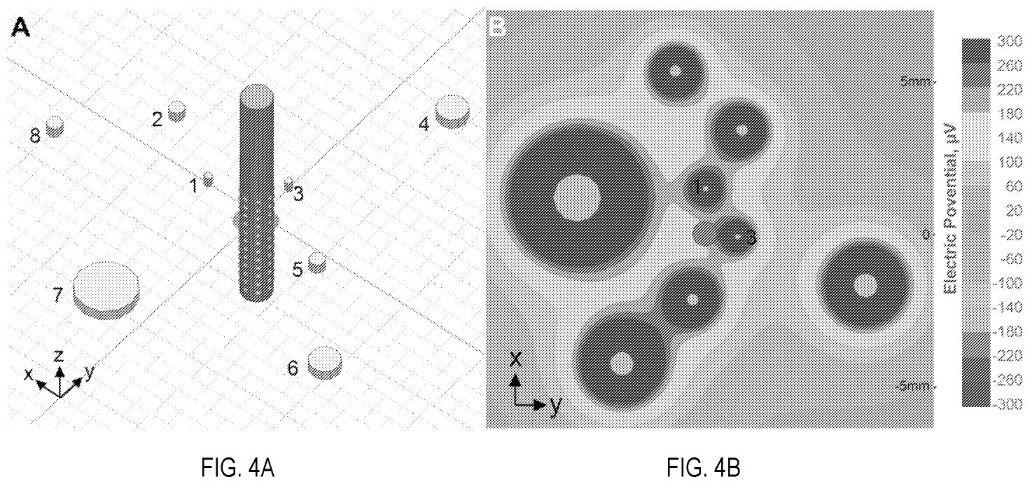

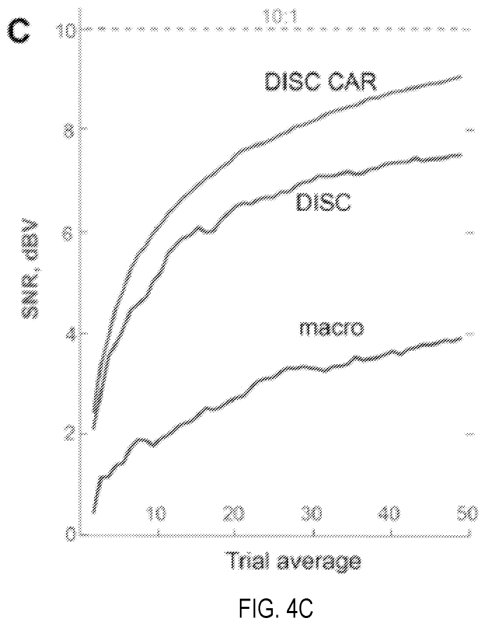

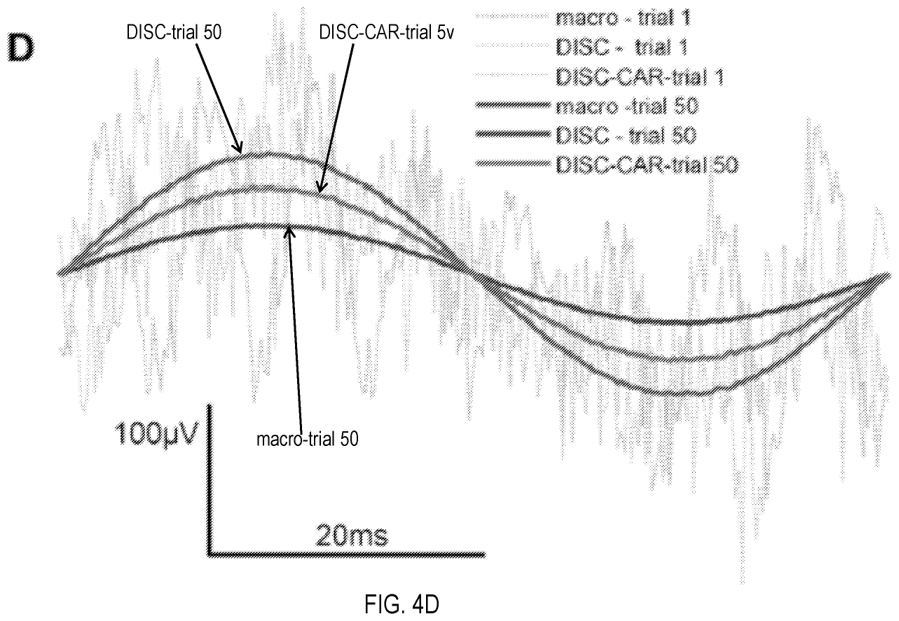

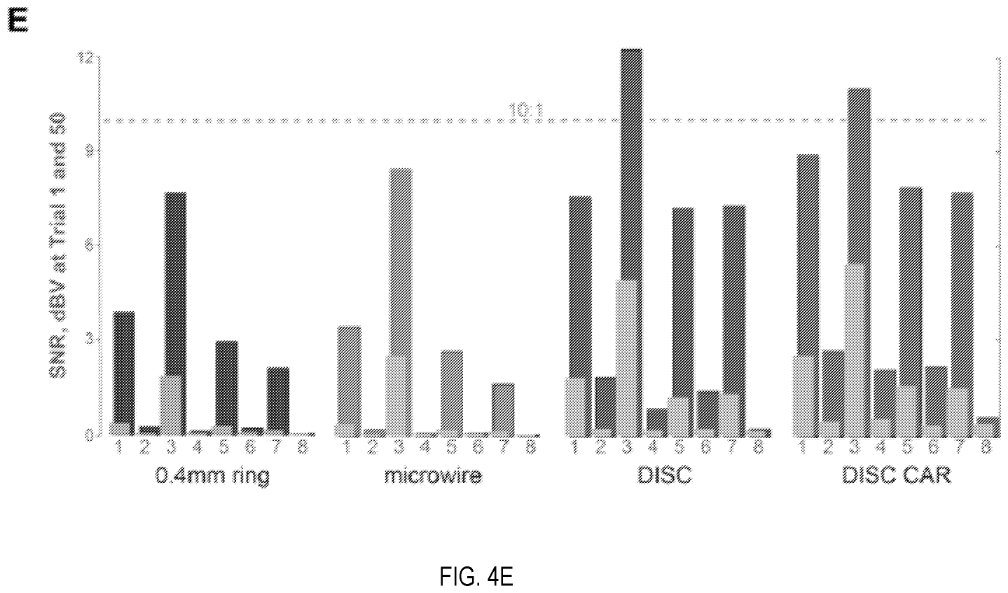

The UTAH ARRAY is a high-density, multi-channel neural sensor used in brain-computer interface applications. The Utah array acts as a high-density bundle of microwires, and microwires offer no substrate shielding because each insulating shaft is small relative to the geometric size of the neural source (FIG. 4).

The directional and scalable (DISC) electrode array described herein provides a number of advantages over conventional neural sensors when recording local field potentials (LFPs).

The DISC electrode array is the first stereotactically delivered microelectrode array designed to separate LFP sources from other simultaneous LFP sources, or any voltage source having an origin that is of a particular size and distance. This is a feature not available with stereo-electroencephalograms (sEEGs) using macro-scale electrodes, microwires, microwire arrays, or ultrafine microelectrode arrays. The DISC electrode array is designed to maximize sensitivity to “mesoscale” neural sources by identifying the source direction from the lead body of the implanted device using a scarcely known phenomenon referred to herein as “substrate shielding.” This is the first application of substrate shielding to produce directional (stereo) measurements of local field potentials in a depth array. To address the accuracy issues of microwires and macroelectrodes, the DISC electrode array detects voltage signals (originating from current sinks and sources) in a direction of tissue within a radius of approximately 0.1-5 millimeters (mm) (i.e., mesoscale).

An array of microelectrodes in the geometry described herein also can simultaneously produce voltage recordings almost identical to ring electrodes or large “directional leads” (segmented ring electrodes) and so can be a complete replacement for macroelectrodes. For example, any circular pattern of microelectrodes when averaged together form a virtual macroelectrode with a height equivalent to the height of the rings of microelectrodes. In another example, microelectrodes can be averaged producing the pattern of large directional electrodes having a known height and arc.

To improve spatial resolution, the DISC electrode array utilizes substrate shielding to provide directional isolation of overlapping LFP signals. This yields a spatial scale that is unique for both neuroscience and brain-computer interfaces (BCIs). The DISC electrode array, includes 2 or 4 linear electrodes (e.g., opposing pairs) with a variable longitudinal span (e.g., 20 mm) and placed on a 0.8 mm cylinder in some examples. Wider diameters provide better shielding but at the cost of displacing and potentially damaging more tissue. The 0.8 mm diameter is advantageous because it is already a safe standard established by neurosurgeons using sEEG and can access deep brain regions.

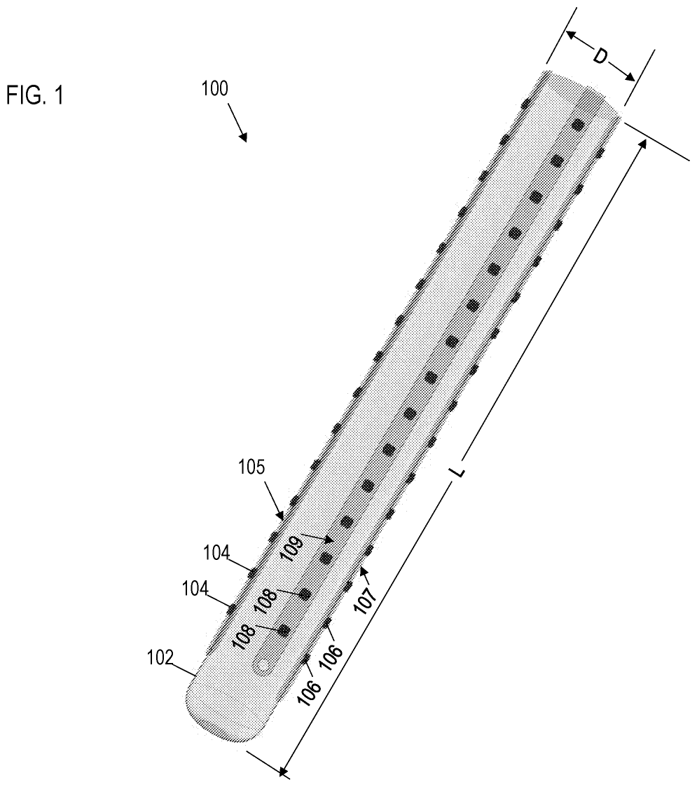

FIG. 1 shows an example DISC electrode array 100. The DISC electrode array 100 is an elongate structure having a diameter D and length L. The diameter D may be in a range of 400-2000 microns. For example, an implementation of the DISC electrode array 100 may have a diameter D of approximately 800 microns (e.g., 800 microns+/−10%). The length L of the DISC electrode array 100 may in a range of 3-150 millimeters in some implementations. The DISC electrode array 100 includes an insulating body 102 and one or more microelectrodes attached to the insulating body 102. The insulating body 102 may be formed of material having an electrical conductivity that is less than 10⁻⁸ siemens per meter. The insulating body 102 is cylindrical in shape in some implementations (i.e., circular in cross-section). Some implementations of the insulating body may be non-circular in cross-section. For example, some implementations of the in insulating body 102 maybe oval, square, hexagonal, octagonal, decagonal, etc. in cross-section.

The microelectrodes 104, 106, and 108 are shown FIG. 1. The microelectrode 104 and the microelectrode 106 are disposed opposite one another on the insulating body 102. That is, the microelectrode 104 and the microelectrode 106 are disposed to form an opposing pair of microelectrodes. The microelectrode 108 is disposed on the insulating body 102 equidistant from the microelectrode 104 and the microelectrode 106. The DISC electrode array 100 may also include a microelectrode opposite the microelectrode 108 forming an opposing pair with the microelectrode 108. Any conducting surface attached to the surface of the insulating body 102 with a conductive path to a low noise amplifier qualifies as a microelectrode. The microelectrodes 104 form a linear subarray 105, the microelectrodes 106 form a linear subarray 107, and the microelectrodes 108 form a linear subarray 108. The linear subarray 105 is opposite (on an opposite side of the insulating body 102 from the linear subarray 107. Similarly, a linear subarray of electrodes may be disposed on the insulating body 102 opposite the linear subarray 109. In the present disclosure, microelectrodes or linear subarrays of microelectrodes are opposite one another if disposed at an angle of 180°±10% from one another about the circumference of the insulating body 102.

Various implementations of the DISC electrode array 100 include different numbers of microelectrodes disposed about the circumference of the insulating body 102 arranged on approximately opposite sides. For example, the DISC electrode array 100 as illustrated in FIG. 1 includes four microelectrodes arranged about the circumference of the insulating body as two opposing pairs. Other implementations of the DISC electrode array 100 may include 2, 6, 8, 10, etc. microelectrodes arranged about the circumference of the insulating body 102 as opposing pairs. Alternatives to opposing pairs of microelectrodes (i.e., an odd number of microelectrodes) is also possible if multiple microelectrode referencing is used as the reference for the recorded voltage. In this case, the centroid of the multiple microelectrode references should be a point opposing the primary microelectrode of interest (referred to as a “common shielded reference” for simplicity).

Implementations of the DISC electrode array 100 include multiple instances of the 104 arranged in a line or row along the length of the insulating body 102, multiple instances of the 106 arranged in a line or row along the length of the insulating body 102, multiple instances of the 108 arranged in a line or row along the length of the insulating body 102, etc. For each instance of each microelectrode disposed on the insulating body 102, an instance of an opposing microelectrode may be disposed on the insulating body 102.

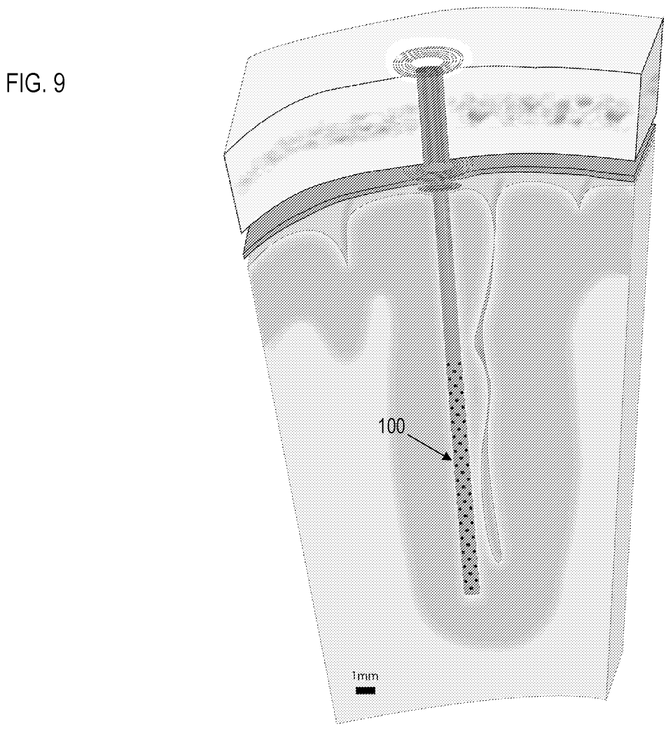

The microelectrodes (e.g., the microelectrodes 104, 106, 108, etc.) may have a diameter in a range of 8-500 microns. The placement of microelectrodes can be limited to the anticipated locations in the brain where neural sources of interest may be found. The spacing (pitch) of microelectrodes near the regions of interest may be arranged in a line or row along the length of the insulating body may be in a range of 200-600 microns. For example, the spacing (pitch) of microelectrodes 104, 106, 108 arranged in a line or row along the length of the insulating body may be about 320 microns in some implementations. In addition to linear arrays of electrodes with opposing linear arrays, electrodes may also be staggered by some arbitrary angle along the longitudinal length. FIG. 9 is an example where every other row is staggered forming diamond patterns of electrodes across the array.

The DISC electrode array 100 may also include a conductor coupled to each of the microelectrodes for conveying electrical signal from the microelectrodes to a processing system coupled to the DISC electrode array 100.

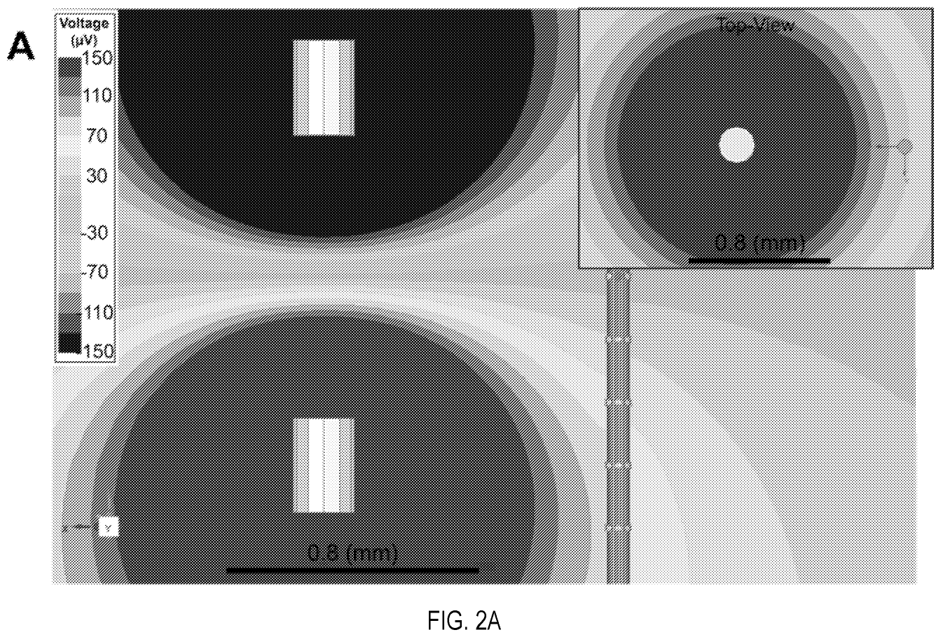

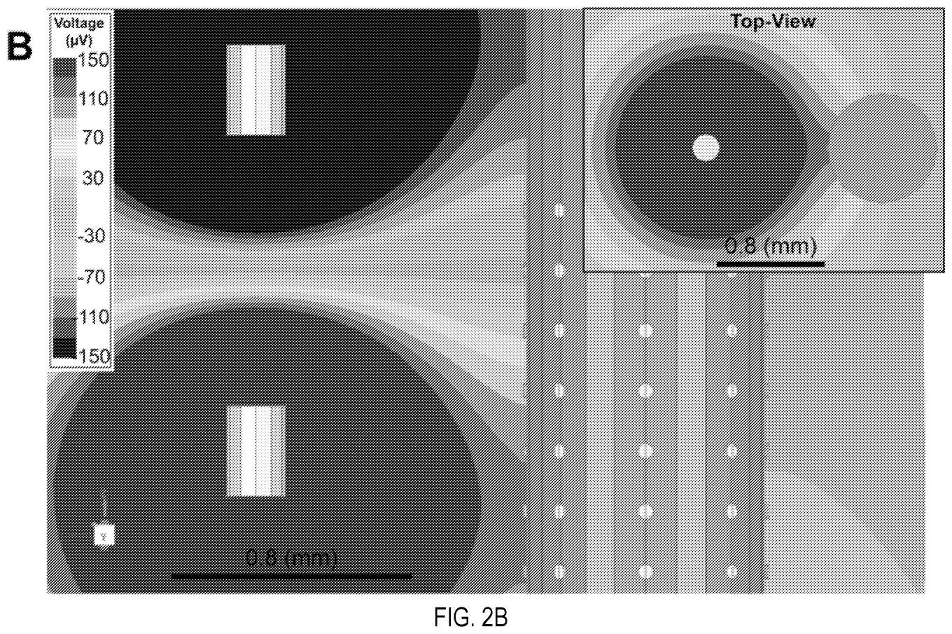

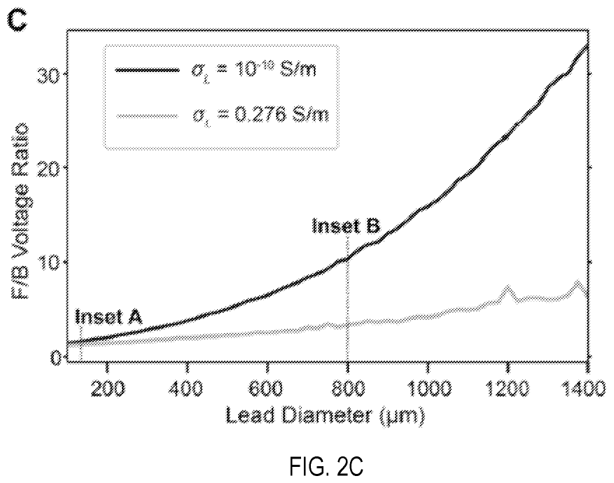

FIGS. 2A-2C illustrate directionality and amplitude as a function of substrate and electrode diameters. The substrates are 65 μm and 800 μm in diameter respectively in FIGS. 2A and 2B, with an electrode on opposite sides of the substrate positioned a fixed distance from a dipole source. All electrodes are independent. Voltage V results from the current source shown to left of the lead body representing layer V pyramidal cells in a virtual cortex (200-μm diameter) created in a finite element model. Voltage is inversely related to distance and perturbed by changes in conductivity σ in space such as are created by the insulating body. FIG. 2C illustrates an ANSYS model of the front to back electrode voltage ratio as a function of diameter (Øₛₕ). When a of the lead body matches local tissue (σ=0.26 S/m), F/B ratio increases due to the increasing distance between front and back electrodes. As shown, substrate shielding magnifies the difference between the front and back electrodes by much more than the previously known method of separating electrodes over a greater distance.

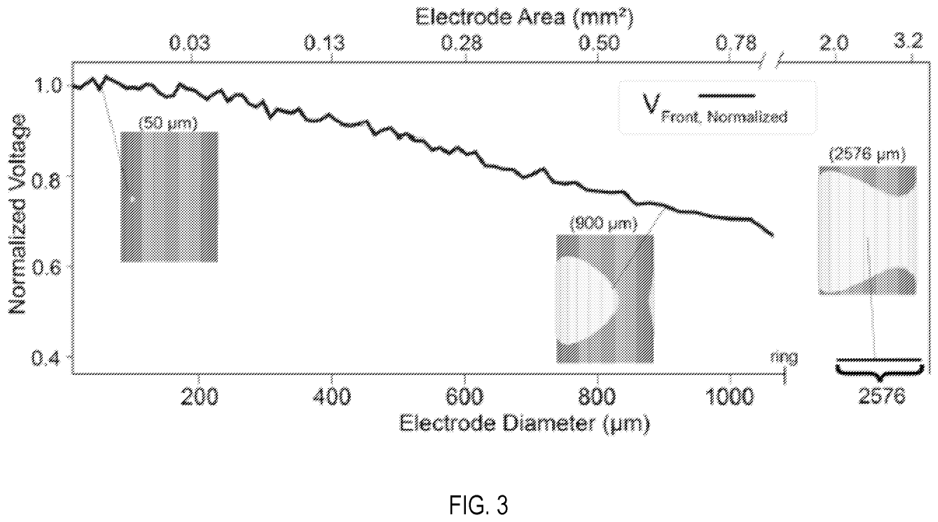

FIG. 3 illustrates normalized voltage amplitude as a function of electrode diameter. The source is the same as in FIGS. 2A and 2B. Attenuation becomes significant beyond about 120 μm. At 1238 μm, a ring forms (e.g., sEEG) and amplitude is attenuated by 60% relative to a microelectrode. Increasing the ring area beyond this point has negligible attenuation.

FIGS. 4A-4E show an electro-quasistatic 3D dipole model demonstrating directional sensitivity in a multi-source configuration. FIG. 4A shows eight simultaneous dipoles (labeled 1-8) modeled in a finite element method with an identical surface boundary current density (only sink is shown, 0.5 mm grid). Three device types are modeled: 1) an implementation of the DISC electrode array 100 (shown in FIG. 4A); 2) microwire; and 3) a 0.4-mm tall ring electrode. FIG. 4B is a voltage heat map through layer V dipoles with sources on a peak current density of 1.39 μA/mm² as described in Murakami, Shingo, and Yoshio Okada, NeuroImage Invariance in Current Dipole Moment Density across Brain Structures and Species: Physiological Constraint for Neuroimaging, NeuroImage 111: 49-58 (2015). The heat map is at the plane intersecting the dipole sinks. FIG. 4C shows signal-to-noise ratio (dBV) for a macro (ring) electrode, a DISC electrode array, and a DISC electrode array with CAR during trial 1 and cumulative trials. FIG. 4D shows waveform examples for 1 and 50 trials for the macro electrode and DISC electrode array when phase locked to Source 1. Sources 2-8 are assigned a random phase and frequency. Noise of 2.7 μVrms and 4.3 μV is assigned to each ring or microelectrode, respectively. FIG. 4E shows a signal-to-noise ratio comparison of the simulated potentials for each source independently phase-locked. The microwire is 65 μm in diameter. Trial 1 is shown in the lighter shade, and trial 50 is shown in the darker shade (avg). This embodiment of DISC electrode array 100 is useful for maximizing SNR for all 8 sources relative to other recording methods simulated.

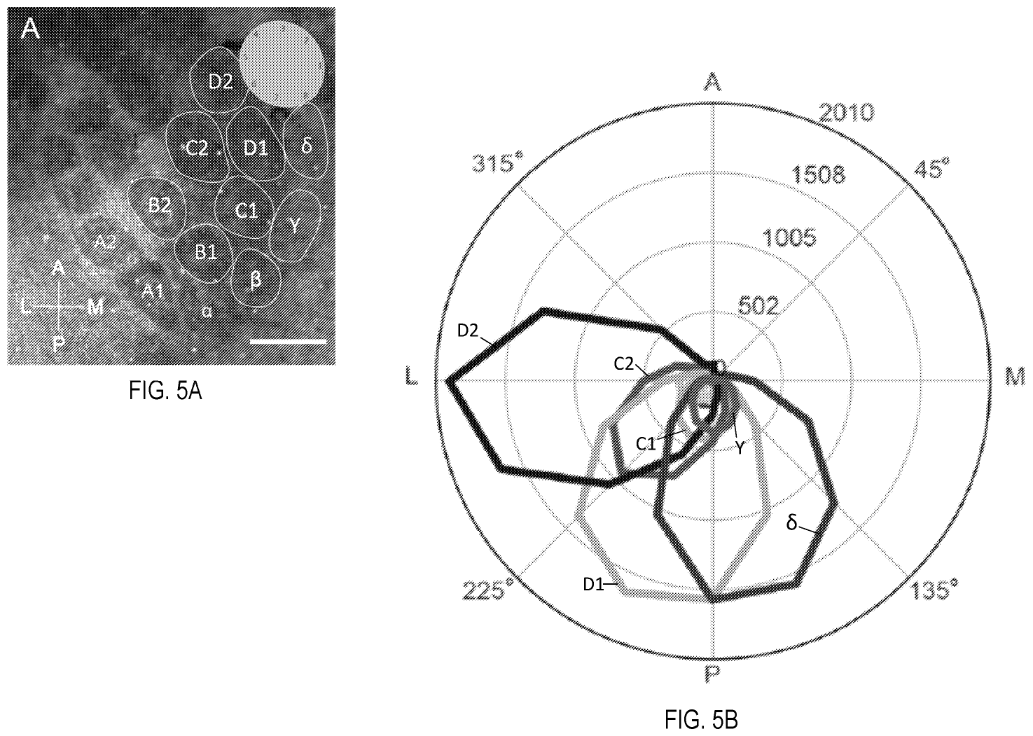

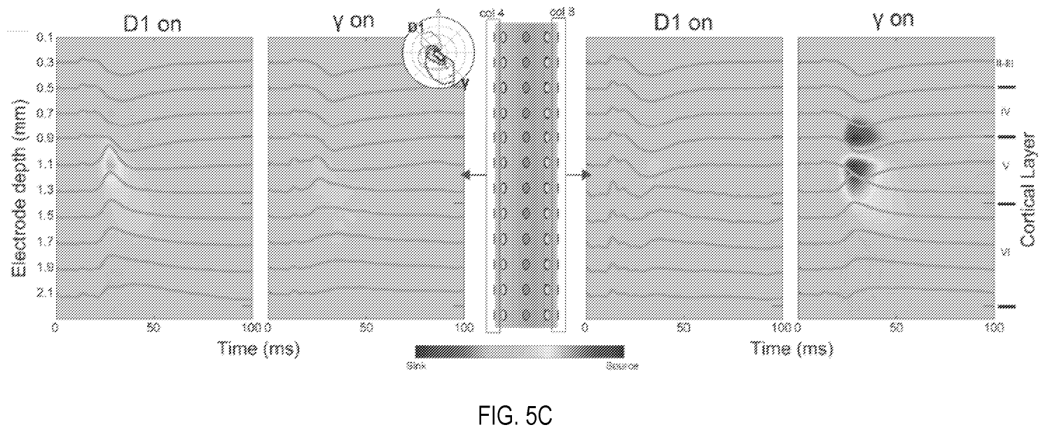

FIGS. 5A-5C illustrate directional sensitivity in a DISC electrode array. FIG. 5A shows an arrangement of nine sources B1, B2, β, C1, C2, D1, D2, δ, and γ relative to a DISC electrode array having eight columns of electrodes. FIG. 5B shows results of finite element modeling using the nine sources and the DISC electrode array. This illustrates why the DISC electrode array 100 is useful for either source separation applications, for example brain computer interfaces, or source localization, for example diagnostic neurosurgery. FIG. 5C shows multi-directional current source density (CSD) from the DISC electrode array when two sources are activated. A first source (D1) is located closest to column 4 of the DISC electrode array, and a second source (Y) is located closest to column 8 of the DISC electrode array (about 180°). FIG. 5C shows distinct amplitude attenuation for a source on the opposite side of the microelectrode column of interest.

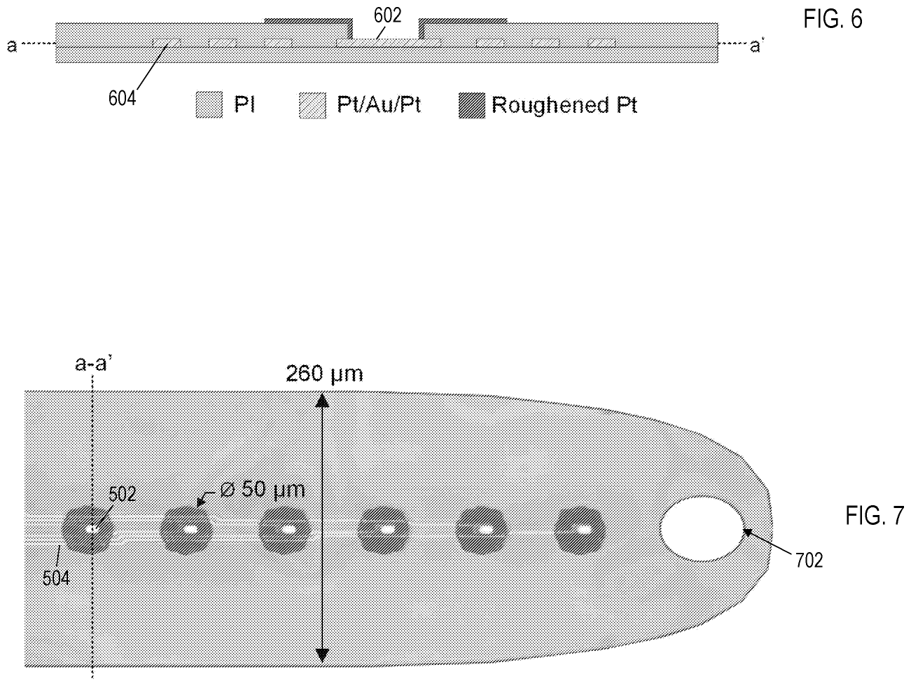

FIG. 6 shows a schematic cross-section of a polyimide linear electrode (PLE) array suitable for use in the DISC electrode array 100. An example microelectrode 602 is shown in the center with insulated interconnects 604 shown traversing parallel to each other in/out of the cross-section.

FIG. 7 shows a micrograph of an example strip of microelectrodes suitable for use in the DISC electrode array 100. Some examples have 80-micron diameter electrodes on a 320-micron pitch. A hole 702 enables easy handling during assembly. Assembly may utilize multiple single-column arrays. Alternatively, not shown, a multi-column microelectrode arrays could also be used to wrap around a cylinder or other geometry and record from other directions.

The DISC electrode array 100 offers the ability to sense LFP sources at the microscale (e.g., current source density), the mesoscale (as demonstrated in FIG. 5B), and at the macroscale. The DISC electrode array 100 is especially novel and useful at the mesoscale (1-5 mm) given that other technologies fail to provide the amplitude and direction resolution of this invention.

At the microscale, it is notable that the DISC electrode array 100 can uniquely measure current source density in tissue from multiple directions simultaneously which is a novel capability useful in the study of laminar communication. Additionally, the amplification of a large diameter substrate makes it possible to record from distant multi-unit or multi-cell action potentials. Most recording arrays for neuroscience applications lack the substrate size to amplify a single-cell or multi-cell source beyond 100-120 μm, however a 1.2-mm diameter DISC substrate will sense a single large pyramidal cell (20-μm diameter, or 2-3 smaller cells) out to 200 μm distance above a 60 μV threshold.

Macroscale recordings from sEEG and ECoG are also highly valuable, and may have advantages to a group model decoder since it uses larger neuron populations and thus may be more predictive of some kinds of behavior (motor movement, speech, etc.) when comparing between patients having such a sensor implanted in similar brain regions. DISC macroscale recordings can be acquired by simply grouping rings of microelectrodes together mimicking properties of a solid ring electrode (albeit with a slightly larger noise floor). The ability to offer multiscale recordings is another important innovation, and it should be expected that any successful BCI solution, especially for speech, will be multi-scale. Further, the safety and simplicity of stereotactic insertion will result in a lower threshold for translation to humans.

In practice, the DISC electrode array 100 may include or be communicatively coupled to circuitry that maximizes the accuracy of source localization of an LPF signal. Such circuitry may include analog circuits, digital circuits, or a processor-based implementation executing software instructions retrieved from memory. In one implementation, analog differential signals acquired from a low-noise amplifier are used to isolate LFP sources from multiple directions.

In one application of the DISC electrode array 100, circuitry incorporated in or communicatively coupled to the DISC electrode array 100 can reconstruct many virtual macroelectrodes through the averaging of signal acquired from a select set of the microelectrodes to generate higher resolution spatial recordings yet still at the macro scale and comparable to conventional SEEG/depth electrodes that sense in 360 degrees or over smaller angular ranges, such as 120, 90, or 45 degrees.

In a system that includes the DISC electrode array 100, a processor may execute software instructions to selectively reference signals acquired from the microelectrodes to maximize the accuracy of source localization of an LFP signal (˜1-350 Hz) or a multi-cell source (˜300-1500 Hz). The optimal referencing is predicted using a biophysical model of the source and an electro-quasistatic model relating current sources to predicted voltages. Such operations are possible only using the DISC electrode array 100.

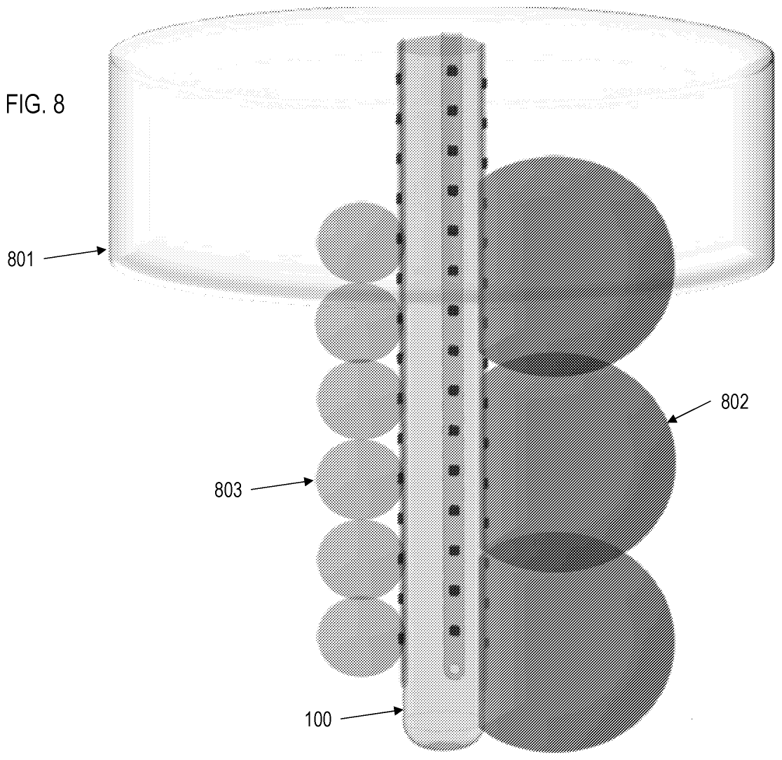

FIG. 8 illustrates examples of scalability achievable using various combinations of microelectrode referencing schemes of the DISC electrode array 100 to produce improved sensitivity in various volumes of surrounding tissue. Various combinations of the microelectrodes of the DISC electrode array 100 are selected and processed to: 1) produce in 801, by averaging signal from all microelectrodes along a selected length of the DISC electrode array 100 output equivalent to sEEG to allow use of standard clinical methods; 2) in 802, isolate semi-local or mesoscale sources; and/or 3) in 803, provide maximum isolation of local sources using current source density analysis which is a form of referencing to only local microelectrodes.

FIG. 9 shows an example DISC electrode array 100 implanted for use as a translatable human BCI. In this alternative arrangement of electrodes, every other row of electrodes is rotated by 45 degrees to effectively create 8 unique columns while only using 4 microelectrodes at any given axial position.

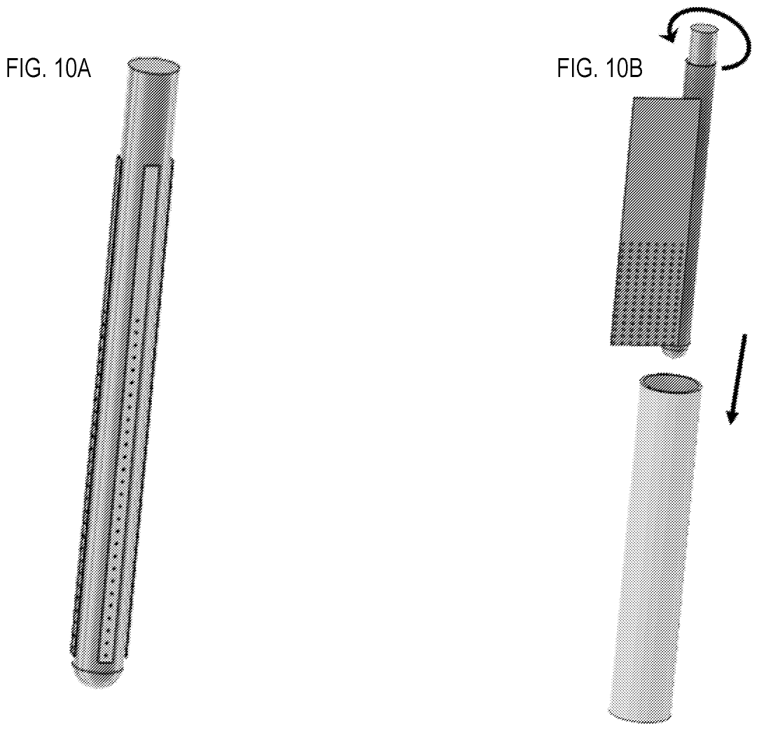

The DISC electrode array 100 can be fabricated using various manufacturing methods. One embodiment includes applying the microfabrication methods of thin film deposition, photolithography, and film etching. Another method includes using medical grade adhesives to attach a thin linear array of microelectrodes (opposing linear arrays of microelectrodes) along the length of an insulating cylinder as shown in FIG. 10A. In one embodiment, the microelectrode arrays may be placed in a hollow cylinder mold with electrodes facing the walls of the mold and then the mold injected with a medical grade insulator, e.g., polyurethane or silicone. Alternatively, a core can be molded or extruded separate from a thin-film array of microelectrodes and the latter is wrapped around the former using a biostable, medical grade adhesive.

In another method, shown in FIG. 10B, for fabricating the DISC electrode assembly 100, a thin (e.g., 20 μm) adhesive sheet is patterned and mounted on the backside of a two-dimensional electrode array (e.g., a 128-channel array). The electrode array and adhesive sheet are wrapped around an insulated substrate (a 432-μm diameter substrate) using heat shrinkable tubing. The tubing is heated to affix the electrode array to the substrate, and the tubing is removed.

Electronic circuitry, including amplifiers, analog-to-digital converters, multiplexers, etc. may be connected to the thin film interconnects of the microelectrodes. In some implementations, such circuitry is disposed within the insulating body 102 (and beneath the skull after implantation of the DISC electrode array 100). In other implementations, the circuitry may be disposed in an insulating body in the skull or outside the skull.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

What is claimed is:

1. A directional and scalable (DISC) electrode array, comprising:
an insulating body comprising an electrically insulating material, and having a length and a diameter, wherein the diameter is at least 400 microns, and the length is greater than the diameter;
a first plurality of microelectrodes disposed along the length of the insulating body; and
a second plurality of microelectrodes disposed along the length of the insulating body opposite the first plurality of microelectrodes.

2. The DISC electrode array of claim 1, further comprising a third plurality of microelectrodes disposed along the length of the insulating body orthogonal to the first plurality of microelectrodes and the second plurality of microelectrodes.

3. The DISC electrode array of claim 2, further comprising a fourth plurality of microelectrodes disposed along the length of the insulating body opposite the third plurality of microelectrodes.

4. The DISC electrode array of claim 3, further comprising a fifth plurality of microelectrodes and a sixth plurality of microelectrodes disposed along the length of the insulating body and opposite to each other.

5. The DISC electrode array of claim 4, further comprising a seventh plurality of microelectrodes and an eighth plurality of microelectrodes disposed along the length of the insulating body opposite to each other and placed orthogonal to the fifth plurality of microelectrodes and the sixth plurality of microelectrodes.

6. The DISC electrode array of claim 1, wherein the diameter of the insulating body is in a range of 400 microns to 2000 microns.

7. The DISC electrode array of claim 1, wherein the diameter of the insulating body is approximately 800 microns.

8. The DISC electrode array of claim 1, wherein the length of the insulating body is in a range of 3 millimeters to 150 millimeters.

9. The DISC electrode array of claim 1, wherein the microelectrodes have a diameter of at least 10 microns.

10. The DISC electrode array of claim 1, wherein two adjacent microelectrodes of the first plurality of microelectrodes are spaced apart in a range of 200-600 microns.

11. The DISC electrode array of claim 1, wherein the insulating body is cylindrical.

12. The DISC electrode array of claim 1, wherein the insulating body is polygonal in cross section.

13. A method for using a directional and scalable (DISC) electrode array, comprising:
acquiring first neuroelectric signals via a first plurality of microelectrodes disposed along a length of an insulating body formed of an electrically insulating material;
acquiring second neuroelectric signals via a second plurality of microelectrodes disposed opposite the first plurality of microelectrodes along the length of the insulating body; and
providing the first neuroelectric signals and the second neuroelectric signals to a processing system for interpretation of neural activity.

14. The method of claim 13, further comprising:
selecting the neuroelectric signals acquired by a single microelectrode of the first plurality of microelectrodes; and
sensing a local field potential source based on the neuroelectric signals acquired by the single microelectrode.

15. The method of claim 13, further comprising:
acquiring third neuroelectric signals via a third plurality of microelectrodes disposed along the length of the insulating body orthogonal to the first plurality of microelectrodes and the second plurality of microelectrodes;
acquiring fourth neuroelectric signals via a fourth plurality of microelectrodes disposed opposite the third plurality of microelectrodes along the length of the insulating body; and
providing the third neuroelectric signals and the fourth neuroelectric signals to the processing system for interpretation of neural activity.

16. The method of claim 15, further comprising:
selecting the neuroelectric signals acquired by the first plurality of microelectrodes, the second plurality of microelectrodes, the third plurality of microelectrodes, and the fourth plurality of microelectrodes disposed along a selected length of the DISC electrode array; and
combining the selected neuroelectric signals to simulate output of a macroelectrode.

17. The method of claim 13, further comprising stereotactically delivering the DISC electrode array through a hole in a skull; wherein the hole is less than 10 millimeters in diameter.

18. A method for fabricating a directional and scalable (DISC) electrode array, comprising:
securing a first linear array of microelectrodes to an insulating body; and
securing a second linear array of microelectrodes to the insulating body opposite the first linear array of microelectrodes.

19. The method of claim 18, further comprising applying an adhesive to one or more of the first linear array of microelectrodes or the insulating body.

20. The method of claim 18, further comprising wrapping a thin film array of microelectrodes around the insulating body to form the first linear array of microelectrodes and the second linear array of microelectrodes.

21. The method of claim 18, further comprising:
positioning the first linear array of microelectrodes in a mold;
positioning the second linear array of microelectrodes in the mold opposite the first linear array of microelectrodes; and
injecting an insulating material into the mold.

22. The method of claim 18, further comprising:
direct printing an insulating substrate onto a sacrificial cylinder;
direct printing a plurality of conductors and electrodes; and
direct printing an outer insulating layer to electrically isolate the plurality of conductors.
